(19) United States
(12) Patent Application Publication
(10) Pub. No.: US 2004/0116938 A1
(43) Pub. Date: Jun. 17, 2004

(54) Medical assembly for introducing a prosthesis and process for holding a prosthesis

(76) Inventors: Simon John Forber, Chasseneuil (FR); Sylvain Masse, Chasseneuil (FR); Damien Sandoz-Othenin, Chasseneuil (FR)

Correspondence Address: YOUNG & THOMPSON, 745 SOUTH 23RD STREET 2ND FLOOR, ARLINGTON, VA, 22202

(21) Appl. No.: 10/637,511

(22) Filed: Aug. 11, 2003

(30) Foreign Application Priority Data
Aug. 9, 2002 (FR) 02 10160

(51) Int. Cl. A61F 11/00

(52) U.S. Cl. 606/108

(57) ABSTRACT

A medical assembly for introducing an expansible prosthesis in a bodily duct includes a tubular sheath opened at a distal end, an elongated element disposed substantially at the centre of the sheath and letting an intermediate radial space between the elongated element and the sheath, and a pusher movable in said tubular sheath. The expansible prosthesis is introduced in the bodily duct by moving between a first holding position in which the prosthesis is at least substantially applied against an internal wall of the sheath, and a second expanded position in which the prosthesis is applied against an internal wall of the bodily duct, the expansible prosthesis passing through intermediate positions in which a proximal portion is inside the sheath and a distal portion is outside the sheath.

The invention relates to a medical assembly for introducing an expansible prosthesis in a bodily duct, and a process for holding an expansible prosthesis in a sheath.

The invention generally applies to expansible prostheses having a tubular shape and which can be introduced in bodily ducts, as vessels, but it is specifically disclosed in the following in reference to a stent which is such an expansible prosthesis.

During surgical operations, surgeons introduce expansible prostheses in specific portions of bodily ducts. Before a surgical operation, a selected prosthesis with a constricted shape is received near an end of a sheath, generally a catheter. During the operation, a surgeon introduces the sheath in a vessel and, when the sheath end has a suitable position, the surgeon operates a pusher to progressively push the prosthesis out of the sheath so that, by self-expanding, the prosthesis applies itself against the internal wall of the vessel.

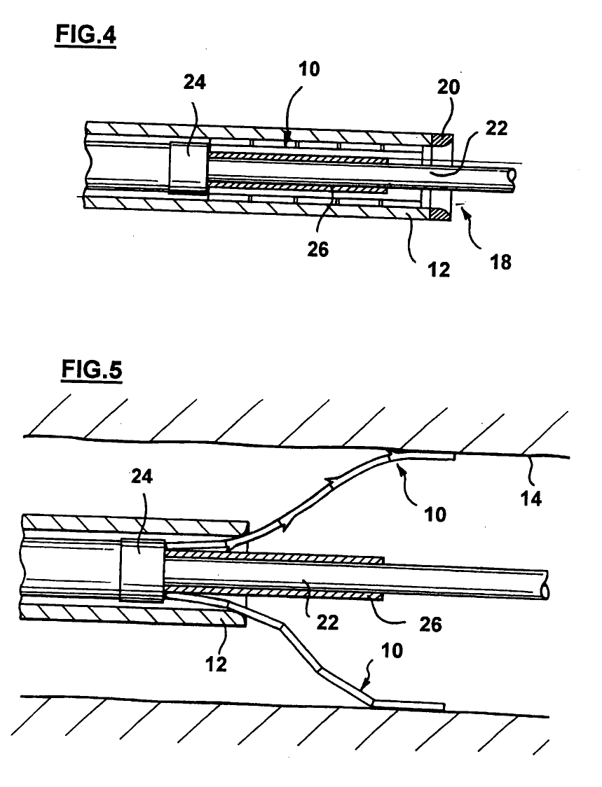

This situation is illustrated by a brief examination of FIGS. 4 and 5 which represent a prosthesis 10 initially received in a sheath 12; the prosthesis is progressively pushed out the sheath and applies itself by expanding against the internal wall of a bodily duct 14.

Such a method for introducing a prosthesis induces an adverse phenomenon which is the early expulsion of the prosthesis from the sheath end, even if the surgeon very progressively pushes the prosthesis.

Especially when the prosthesis is short, for example has a length of 20 mm, and is to be received in a duct having an internal diameter corresponding to the top portion of the duct diameter range for which the prosthesis is recommended, the prosthesis is expelled from sheath end and does not take the desired position.

This expulsion phenomenon is very detrimental because the prosthesis may take an erroneous position and be jammed. Further, it is not possible to retrieve the prosthesis inside the human body. So, it is very important to suppress this phenomenon.

This expulsion phenomenon can be understood by the following explanation which does not limit the invention.

When the prosthesis is entirely inside the sheath (FIG. 4), it applies itself against the internal wall of the sheath, and does not tend to move along the sheath. The expansion force which applies the prosthesis against the internal wall of the sheath generates a high friction force (F_{T} in a FIG. 1) against the internal surface, and this friction force opposes to prosthesis motion.

However, as soon as a portion of the prosthesis is outside the sheath end, this portion spreads radially outside, and the prosthesis elastically bends. As the prosthesis has a longitudinal rigidity and is in contact against the internal edge of the opening at the sheath end, the portion just inside the opening of the sheath end tends to be radially distant from the internal surface of the sheath.

At the contact point between the prosthesis and the internal edge of the opening of the sheath, the tangent to the prosthesis is inclined by an angle α with the sheath axis. As the prosthesis bends, a reaction force F_{C} (as shown by FIG. 1) is applied by the internal edge. This force F_{C} due to bending has a component F_{T} parallel to sheath axis. This component F_{T} has an opposite direction to holding force F_{R} direction due to friction of the prosthesis portion in contact with the internal sheath wall.

When the surgeon goes on pushing the prosthesis out of the sheath, the contact area between the prosthesis and the internal sheath wall decreases, and so the holding force F_{T} progressively decreases. In contrast, component F_{T} of the bending force keeps a nearly constant value, and it soon appears a situation in which the friction or holding force F_{R} becomes less than the component F_{T} of the bending reaction force. From this moment, the resultant of the forces applied to the prosthesis is directed outside the sheath end and the prosthesis is expelled.

If a significant portion of the prosthesis is yet applied against the wall of bodily duct, a retaining force is exerted on the prosthesis by the bodily duct and the resultant of all forces prevents expulsion.

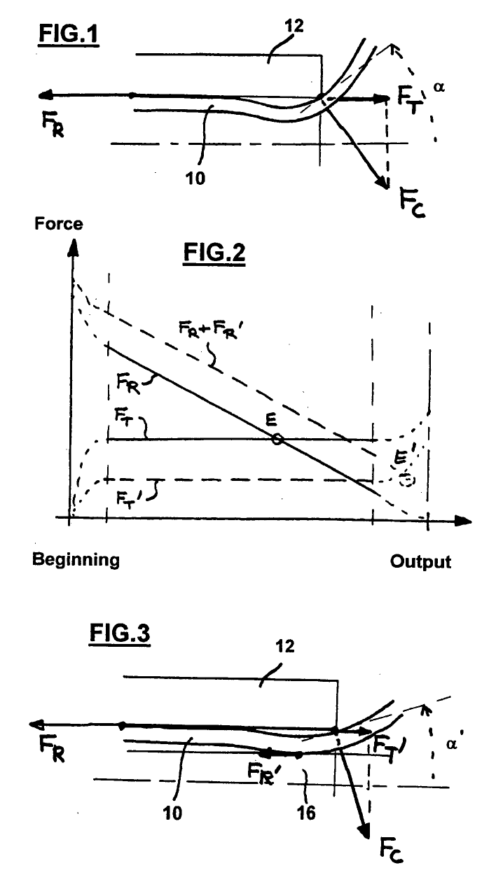

This phenomenon may be best understood in reference to FIG. 2 diagram.

In FIG. 2, continuous line F_{R} illustrates holding force variation due to friction of the prosthesis inside the sheath. This force linearly decreases between the beginning of pushing (initial pusher position), that is when prosthesis is completely inside the sheath, and the end of pushing when all the prosthesis is outside the sheath. Close by these two extreme positions, secondary processes may be present, but they are not significant for understanding the expulsion phenomenon.

Pulling force F_{T} is the component of prosthesis bending force F_{C} which is parallel to the sheath axis and is directed outwards. This force F_{T} is shown as substantially constant because the prosthesis has frequently the same longitudinal rigidity and the same bending strength on all its length.

Holding force F_{R} becomes equal to pulling force F_{T} at point E. The prosthesis is expelled at or near point E. If point E is near the sheath end, the expulsion phenomenon is practically insignificant because the prosthesis is yet well held by the bodily duct. However, the more point E is near the beginning of pushing, and the more abrupt is the prosthesis expulsion.

Several systems are yet known for solving the problem of preventing this expulsion phenomenon.

U.S. Pat. No. 6,120,522 discloses a system in which the prosthesis expulsion is delayed by incorporating an elastic membrane at the sheath end. The elastic membrane applies friction and holding forces for preventing prosthesis expulsion.

International Patent Application WO 99/49 812 discloses a system in which the proximal portion of a prosthesis is retained by blades projecting from a special pusher. In this system, the prosthesis can separate itself from pusher only when pusher is yet outside the sheath. The pusher has a shape specific to the prosthesis because he has to retain the prosthesis on two sides.

International Patent Application WO 01/17 458 discloses a system in which expulsion phenomenon is prevented because a sleeve wedges the prosthesis between itself and the sheath before the expulsion moment. The surgeon has to apply a great force for moving prosthesis, because the prosthesis is wedged between two members.

The invention relates to a solution of said problem which is completely different from solutions of said known systems. The principle of this solution according to the invention is then disclosed in reference to FIG. 3.

According to the invention, two phenomena are used for preventing the prosthesis expulsion: the first one is the reduction of the force which tends to expel the prosthesis, and the second is addition of a supplementary holding force to a prosthesis inside the sheath.

As shown by FIG. 3, an internal element designated by numeral 16 partially compresses radially outwards the prosthesis portion which is bended inside the sheath near its end. This element 16 restricts the prosthesis bending which is smaller than illustrated by FIG. 1.

The partial compression by element 16 has two results. The first one is a reduction of the prosthesis tangent angle of sheath 12; the angle α of FIG. 1 becomes the smaller angle α′, so that the pulling component F_{T}′ of bending force F_{C} in parallel direction with the sheath axis is reduced, and the force pulling the prosthesis outwards is reduced. The second result is that the contact between the internal element 16 which applies the partial compression to the prosthesis generates a supplementary holding force F_{R}′ at the contact surface, this force being a friction or catching force.

As illustrated by FIG. 2, on the one hand, the pulling component F_{T}′ is smaller than the pulling component F_{T} and, on the other hand, the holding force F_{R} due to friction inside the sheath is increased by a supplementary holding force F_{R}′. The total holding force is then F_{R}+F_{R}′.

FIG. 2 shows that point E′ at which the increased holding force F_{R}+F_{R}′ becomes equal to the reduced pulling force F_{R}′ is substantially shifted outside the sheath, and the expulsion phenomenon is practically prevented.

The schematic example disclosed in reference to FIGS. 2 and 3 corresponds to a small friction force F_{R}′ between the internal element 16 and the prosthesis. However, this force may be controlled by several means as disclosed in the following, and it may be increased to completely prevent expulsion of the prosthesis.

The solution according to the invention relates essentially to reducing the pulling force by reducing the tangent angle, and secondarily to increasing the holding force.

The invention applies the above solution to a medical assembly for introducing an expansible prosthesis in a bodily duct, and to a process for holding an expansible prosthesis in a sheath.

Accordingly, the invention relates to a medical assembly for introducing an expansible prosthesis in a bodily duct, said assembly being of the kind which includes a tubular sheath opened at a distal end, an elongated element disposed substantially at the centre of the sheath and letting an intermediate radial space between said elongated element and said sheath, and a pusher movable in said tubular sheath, said expansible prosthesis being introduced in said bodily duct by moving between a first holding position in which said prosthesis is at least substantially applied against an internal wall of said sheath due to its expansion force, and a second expanded position in which said prosthesis is applied against an internal wall of said bodily duct, said expansible prosthesis passing through intermediate positions in which a proximal portion of said prosthesis is inside said sheath and a distal portion is outside said sheath beyond distal end of said sheath; according to the invention, the radial dimension of said intermediate space between said sheath and said elongated element is greater than the radial dimension of said prosthesis received inside said sheath so that, independently of tolerances applied to dimensions of said prosthesis, said sheath and said elongated element, said prosthesis is not in contact with said elongated element when prosthesis is in its first position, and the longitudinal rigidity of said prosthesis and the radial dimension of said intermediate space present a relation such that said longitudinal rigidity of said prosthesis is sufficiently high and said radial dimension of said intermediate space is sufficiently small for, in one of said intermediate positions in which a portion of said prosthesis is beyond distal end of said sheath, a portion of said prosthesis inside said sheath is apart from said sheath wall and is in contact with said elongated element near said distal end of said sheath.

Preferably, the external surface of said elongated element includes, at least at a position to be in contact with said internal surface of said prosthesis, a member for creating a force for holding said prosthesis in a direction parallel to said elongated element axis.

In an embodiment, said member for creating a force for holding is a member for creating a friction force, and said member is selected between an elastomer material, an adhesive material, a soft material and a rugose material.

In another embodiment, said member for creating a force for holding is a member for creating a catching force, and said member is selected between cavities of said elongated member surface, such as series of grooves or a spiral cavity, and projections, such as a series of wings or a spiral projection. Preferably, said cavities and projections have rounded edges.

In a preferable embodiment, said movable pusher is integrated with said elongated element.

The invention also relates to a process for holding an expansible prosthesis partially projecting from a sheath end and spread radially outside said sheath end and contracted radially inside said sheath end, a tangent to said prosthesis at its point of contact with said sheath end forming an angle with said sheath axis; according to the invention, said process includes partially compressing radially outwards said portion contracted radially inside said sheath end, said partial compression reducing said angle of said tangent to the prosthesis with said sheath axis at the contact point with said sheath end.

Preferably, said partial compression induces a holding force in said contact area of said portion contracted radially inside said sheath end and an internal contact member. Preferably, said partial compression is applied by means selected between means generating a friction force and means generating a mechanical catching holding force.

Other features of the invention may be better understood by reading the following description of embodiments, in reference to drawings in which:

FIG. 1, yet described, is a diagram useful for describing the problem solved by the invention;

FIG. 2 is a diagram useful for illustrating behaviour of prior art and of the invention;

FIG. 3 is the diagram similar to FIG. 1, but illustrating the invention;

FIG. 4 is a longitudinal section of an embodiment of medical assembly according to the invention;

FIG. 5 illustrates FIG. 4 assembly in a stage where a prosthesis is nearly out of said assembly for introducing;

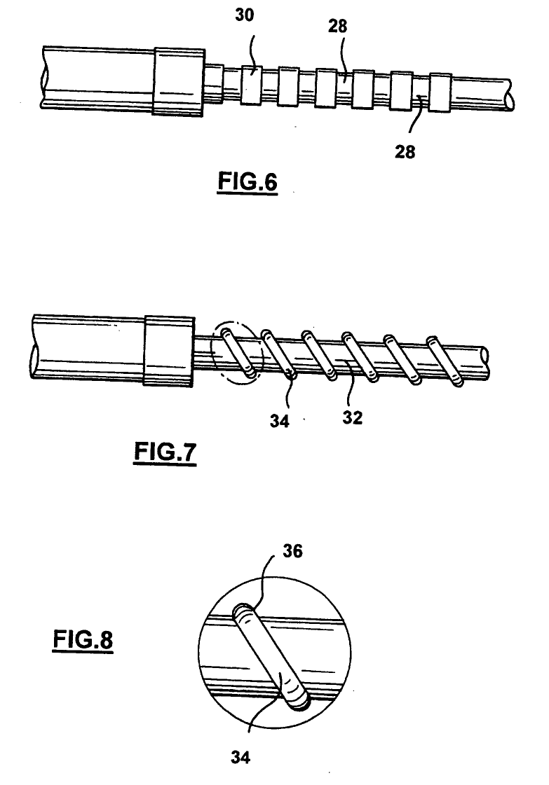

FIGS. 6 and 7 show two embodiments of mechanical catching means useful according to the invention; and

FIG. 8 is an enla rged representation of the circled detail of FIG. 7.

FIG. 4 illustrates an example of medical assembly for introducing a prosthesis in a bodily duct. Prosthesis 10 is confined inside a sheath 12 having an end opening 18 defined in this embodiment by a ring 20 constituted by a radio-opaque material useful for observing surgical operation evolution.

An elongated element 22 is at the sheath centre and is integrated which a pusher 24 which is in contact with prosthesis 10 proximal end. Elongated element 22 is for example a catheter. Elongated element 22 has a surface coating 26. In the disclosed example, said coating is constituted by a polyurethane elastomer. However, it may be constituted by several materials for generating a friction holding force. The material effect may be the result of its friction coefficient, its tackiness, its springiness, its softness and/or its rugosity. The men in the art can easily choose a suitable material in view of the desired result, as disclosed in the following in reference to a medical assembly construction.

FIG. 5 illustrates a stage near the end of prosthesis positioning. Distal end of prosthesis 10 is in contact with internal wall of duct 14, but its proximal end is still inside the sheath, in contact with the pusher. In the final stage, the behaviour disclosed in reference with FIG. 3 is yet finished because the proximal end of prosthesis is yet apart from internal sheath wall. Holding force includes only the friction force against the internal coating 26.

FIGS. 4 and 5 illustrate a coating 26 constituted by a material creating a holding force by friction. It is also possible to create a holding force by catching. Prostheses used in embodiments of the invention are frequently stents constituted by tortuous threads defining openings and having many edges easily catching every unevenness of the internal element.

FIG. 6 discloses a first example of unevenness obtained by forming grooves 28 between lands 30 at external surface of an elongated element.

FIG. 7 discloses an embodiment in which projections are constituted by a spiral at the surface of an elongated element. Spiral 32 forms windings 34 having preferably rounded edges 36 as shown by FIG. 8.

The edges of a prosthesis can temporarily catch projections constituted by spiral windings 34 or edges of lands 30 between grooves 28.

According to the invention, there is a relation between the longitudinal rigidity of the prosthesis and the radial dimension of the intermediate space between sheath and elongated element. As the relation implies many variables and as members have very small dimensions (external diameter of catheter constituting sheath is frequently of about 2 to 2.5 mm), this relation has to be determined empirically. Determination of this relation according to the invention is then disclosed.

When the prosthesis to be introduced and the corresponding sheath have been selected, the prosthesis is partially introduced in the sheath in the position disclosed by FIG. 1, preferably near the expulsion conditions.

It is then easy to determine the following dimensions : internal diameter D1 of sheath, internal diameter D2 of prosthesis at a position where prosthesis is in contact with sheath, and internal diameter D3 of prosthesis at the position where prosthesis is the most distant from internal wall of sheath, toward sheath axis.

Tolerances on dimensions of prosthesis, sheath and elongated element are then accounted, so that it is easy to determine what should be the kind and the diameter of the elongated element to use with the selected prosthesis and sheath and in view of the specifications of the particular application.

Firstly, the external diameter D of elongated element has to be smaller than diameter D2, so that the elongated element is not in contact with prosthesis at its first position (against internal wall of sheath).

Secondly, in accordance with the invention, the elongated element has to have an external diameter D greater than internal diameter D3 of prosthesis at the point where it is the most distant from internal wall of sheath.

The external diameter D of the elongated element 16 (catheter with possibly a coating) and its surface features (coating or grooves or projections) are then determined. The diameter is selected between the two said diameters D2 and D3 in accordance with the desired reduction of the pulling force. The more the diameter D is near diameter D2 and the more is the reduction of pulling force.

The surface features of the elongated element are defined in accordance with the desired holding force. Supplementary holding force is substantially determined by the holding force generated by contact between the prosthesis and elongated element materials.

If the prosthesis has a great length, it may be favourable to do not excessively increase the holding force, and the reduction of pulling force is preferable. If the prosthesis is short, it is favourable to increase the holding force by creating a high friction and/or catching force at interface between prosthesis and elongated element.

Tests executed with medical introduction assemblies according to the invention have shown that it is possible to completely prevent shocks when the prosthesis goes out of the sheath. Prosthesis jams are moreover never observed. So, a prosthesis is progressively introduced and the surgeon has a complete control during the prosthesis introduction.

1. A medical assembly for introducing an expansible prosthesis in a bodily duct, said assembly being of the kind which includes
a tubular sheath opened at a distal end,
an elongated element disposed substantially at the centre of the sheath and letting an intermediate radial space between said elongated element and said sheath, and
a pusher movable in said tubular sheath,
said expansible prosthesis being introduced in said bodily duct by moving between a first holding position in which said prosthesis is at least substantially applied against an internal wall of said sheath due to its expansion force, and a second expanded position in which said prosthesis is applied against an internal wall of said bodily duct, said expansible prosthesis passing through intermediate positions in which a proximal portion of said prosthesis is inside said sheath and a distal portion is outside said sheath beyond distal end of said sheath,
wherein the radial dimension of said intermediate space between said sheath and said elongated element is greater than the radial dimension of said prosthesis received inside said sheath so that, independently of tolerances applied to dimensions of said prosthesis, said sheath and said elongated element, said prosthesis is not in contact with said elongated element when said prosthesis is in its first position, and
wherein longitudinal rigidity of said prosthesis and radial dimension of said intermediate space present a relation such that said longitudinal rigidity of said prosthesis is sufficiently high and said radial dimension of said intermediate space is sufficiently small for, in one of said intermediate positions in which a portion of said prosthesis is beyond distal end of said sheath, a portion of said prosthesis inside said sheath is apart from said sheath wall and is in contact with said elongated element near said distal end of said sheath.

2. A medical assembly for introducing an expansible prosthesis in a bodily duct according to claim 1, wherein the external surface of said elongated element includes, at least at a position to be in contact with said internal surface of said prosthesis, a member for creating a force for holding said prosthesis in a direction parallel to said elongated element axis.

3. A medical assembly for introducing an expansible prosthesis in a bodily duct according to claim 2, wherein said member for creating a force for holding is a member for creating a friction force.

4. A medical assembly for introducing an expansible prosthesis in a bodily duct according to claim 3, wherein said member for creating a friction force is selected between an elastomer material, an adhesive material, a soft material and a rugose material.

5. A medical assembly for introducing an expansible prosthesis in a bodily duct according to claim 2, wherein said member for creating a force for holding is a member for creating a catching force.

6. A medical assembly for introducing an expansible prosthesis in a bodily duct according to claim 5, wherein said member for creating a catching force includes cavities of said elongated member surface selected between a series of grooves and a spiral cavity.

7. A medical assembly for introducing an expansible prosthesis in a bodily duct according to claim 5, wherein said member for creating a catching force includes projections of said elongated member surface selected between a series of wings and a spiral projection.

8. A medical assembly for introducing an expansible prosthesis in a bodily duct according to claim 1, wherein said movable pusher is integrated with said elongated element.

9. A process for holding an expansible prosthesis partially projecting from a sheath end and spread radially outside said sheath end and contracted radially inside said sheath end, a tangent to said prosthesis at its point of contact with said sheath end forming an angle with said sheath axis, wherein said process includes the step of partially compressing radially outwards said portion contracted radially inside said sheath end, said partial compression reducing said angle of said tangent to the prosthesis with said sheath axis at the contact point with said sheath end.

10. A process for holding an expansible prosthesis according to claim 9, wherein said partially compressing step induces a holding force in said contact area of said portion contracted radially inside said sheath end with an internal contact member.

11. A process for holding an expansible prosthesis according to claim 9, wherein said partial compression is applied by means selected between means generating a friction force and means generating a mechanical catching holding force.
